(19) United States
(12) Patent Application Publication
(10) Pub. No.: US 2026/0338749 A1
(43) Pub. Date: Oct. 29, 2026

(54) SYSTEM AND METHOD FOR ANALYSIS OF EMISSIONS

(71) Applicant: Michael D. Crouch, Baton Rouge, LA (US)

(72) Inventor: Michael D. Crouch, Baton Rouge, LA (US)

(21) Appl. No.: 19/486,583

(22) PCT Filed: May 31, 2024
(86) PCT No.: PCT/US2024/031853

§ 371 Date: Nov. 21, 2025

Related U.S. Application Data
(60) Provisional application No. 63/470,652, filed on Jun. 2, 2023.

(51) Int. Cl. G01N 33/00; G01N 1/22; G01N 30/02; G01N 30/72; G01P 5/02; G01P 13/04

(52) U.S. Cl. CPC G01N 33/0031; G01N 1/22; G01N 30/7206; G01N 33/0062; G01P 5/02; G01P 13/045; G01N 2030/025

(57) ABSTRACT

This invention provides systems and methods for obtaining vectored air analyte data, which may assist in the determination of a source or sources of emissions.

WO

### TECHNICAL FIELD

This invention relates to systems and methods for obtaining vectored air analyte data, which may assist in the determination of a source or sources of emissions.

### BACKGROUND

Air quality is an increasingly important environmental and health issue. Consequently, it is more imperative to be able to effectively monitor air quality and to determine the sources of emissions (e.g., benzene, methane, and particulates). Determining the source of emissions is an ongoing issue because of the environmental regulations to which industry must adhere, as well as for governmental and other environmental air quality monitoring agencies.

It is important that emissions resulting from events such as leaking valves, leaking storage tanks, loading activities, and other manufacturing activities as well as spillage or accidental releases be identified as quickly as possible for faster evaluation and any needed mitigation. Identifying the source and measuring the relative concentrations of species in emissions are necessary for the determination of acute, chronic, and environmental exposure as well as for meeting environmental regulatory and permit requirements.

Several products and methods to determine sources of air emissions and leaks are available. Some methods involve a human being walking around with a handheld sensor to test various locations to determine releases and their sources. Other methods use handheld infrared camera systems to determine areas from which emissions (leaks) are occurring. Still other methods use combinations of air sensors and high-precision gas sensors, and sometimes weather data, to feed data into one or more models to determine a source of emission.

There remains a need for a method that directly measures data and correlates the data with an emission source.

### SUMMARY OF THE INVENTION

This invention provides methods to determine, record, and display the direction from which one or more substances (emissions) is traveling at the time of measurement. The systems and methods of this invention can determine a source or sources of one or more substances (emissions) when the substance is released, so that the emissions can be addressed. Displaying data from each air sample is an advantage provided by the systems and methods of this invention, especially when the data is made available in real time via a repository that allows real time access to the data.

An embodiment of this invention is a system for obtaining vectored air analyte data, which system comprises:
one or more detector arrays, each detector array having a location and each detector array comprising:
   i) an air analyzer comprising an air intake configured to obtain respective air samples over time, the air analyzer being configured to analyze the air samples obtained over time by the air intake to produce air sample analyte information,
   ii) an anemometer co-located with the air analyzer, the anemometer being configured to measure wind velocity and wind direction concurrent with obtaining the air samples, and
   iii) a transmitter for transmitting detector array data comprising the air sample analyte information, time at which each air sample was obtained, wind speed and wind direction at each time at which each air sample was obtained, and information indicative of the location of the detector array;
a repository in which the detector array data are stored upon receipt from the transmitter; and
a data processor having machine readable logic instructions which when executed by a central processing unit processes at least a portion of the detector array data so as to generate one or more signals convertible into one or more display images showing the detector array data in human-perceivable form.

Another embodiment of this invention is a method for assembling a system for obtaining vectored air analyte data. Other embodiments of the invention include methods for obtaining vectored air analyte data.

These and other embodiments and features of this invention will be still further apparent from the ensuing description and appended claims.

### BRIEF DESCRIPTION OF THE DRAWINGS

FIG. 1 is a schematic of a preferred system of the invention.

FIG. 2 is a view of a computer screen as a display for the air analyte data in accord with one embodiment of the invention.

FIG. 3A is a representation of air analyte data over a period of time for two detector array locations overlaid on a map of the facility where the detector arrays are located in accord with the embodiment of FIG. 2; the series graphs to the left are for a specific substance at several detector array locations.

FIG. 3B is an enlargement of the series of graphs shown in FIG. 3A.

FIG. 3C is an enlargement of the vectors overlaid on a map of a chemical plant shown in FIG. 3A.

The figures illustrate embodiments of specific aspects of the invention, and are not intended to impose limitations on the scope of the invention.

### FURTHER DETAILED DESCRIPTION OF THE INVENTION

As used throughout this document, the words “emission” and “emissions” refer to a substance or substances that are being measured in the practice of this invention. These substances are also sometimes referred to as contaminants or pollutants. Typically, the substance or substances pose health risks to humans, and are often regulated by governmental bodies. The substance(s) can be one or more chemicals, particulates, or other species that an air analyzer can detect.

The systems of the invention comprise one or more detector arrays, a transmitter for transmitting detector array data, a repository in which detector array data are stored after receipt from the transmitter, and a data processor having machine readable logic instructions which when executed by a central processing unit processes at least a portion of the detector array data so as to generate one or more signals convertible into one or more display images showing the detector array data in human-perceivable form.

Each detector array has a location, which location can be referenced to a convenient fixed point, or, preferably, the location is a geographic position. Each detector array is comprised of an air analyzer, an anemometer co-located with the air analyzer, and a transmitter for transmitting detector array data comprising the air sample analyte information, time at which each air sample was obtained, wind speed and wind direction at each time at which each air sample was obtained, and information indicative of the location of the detector array.

Each air analyzer comprises an air intake. The air intake is configured to obtain respective air samples over time, for analysis by the air analyzer. The air analyzer is configured to analyze the air samples obtained over time by the air intake to produce air sample analyte information. The air intakes can be passive or active as desired.

Each air analyzer comprises one or more substance-specific sensors and/or a multi-substance analyzer. In preferred embodiments, each air analyzer comprises one or more substance-specific sensors or a multi-substance analyzer. When the air analyzer comprises one or more substance-specific sensors, the number and type of substance-specific sensors can vary as needed in a specific location (e.g., within a chemical plant) or specific situation (e.g., an event such as a train accident causing a chemical leak). Substance-specific sensors include, but are not limited to, carbon monoxide sensors, carbon dioxide sensors, methane sensors, and particulate sensors. In some embodiments, the substance-specific sensor is selected from a carbon monoxide sensor, a carbon dioxide sensor, a methane sensor, and/or a particulate sensor. In some embodiments in which air analyzer comprises more than one substance-specific sensor, the substance-specific sensors may be in the form of a sensor array. Multi-substance analyzers are typically gas chromatographs (GCs) or multi-substance sensors; gas chromatographs are sometimes preferred. A gas chromatograph has a detector type that can be, but is not limited to, a photoionization detector, an electron capture detector, a flame ionization detector, and a mass spectrometer detector. Preferred are gas chromatographs with a mass spectrometer detector (GC-MS). In some particular embodiments, an air analyzer is an environmental gas chromatograph, preferably an environmental gas chromatograph that is mobile and operates in the field. One such environmental gas chromatograph is available from ENMET, LLC, and is equipped with a photoionization detector, and the environmental gas chromatograph is often used to detect aromatic hydrocarbons such as benzene, toluene, ethyl benzene and xylenes.

Each anemometer is co-located with a respective air analyzer, and each anemometer is configured to measure wind velocity and wind direction concurrent with obtaining each air sample.

The air analyzer(s) and anemometer(s) can be commercially-available air analyzers and anemometers. Preferred anemometers are automatic, mechanical, or solid-state anemometers. In some embodiments, a suitable mechanical anemometer is a Heavy Duty Wind Monitor available commercially from R. M. Young Company.

Each detector array has a transmitter for transmitting detector array data comprising the air sample analyte information, time at which each air sample was obtained, wind speed and wind direction at each time at which each air sample was obtained, and information indicative of the location of the detector array. The transmitter provides for transmission of the detector array data to a repository. The transmitter includes wired and wireless transmission; wireless transmission may not be feasible in some locations. Preferably, the transmitter for transmitting detector array data comprises a wireless transmitter configured to transmit the detector array data to a global computer network in communication with the repository for storage.

The repository in which the detector array data are stored can be any storage medium capable of storing computer data, including computer hard drives, solid state storage devices, flash drives, RAM, ROM, removable computer data storage media, local network servers, and cloud-based data storage systems. Preferred repositories include computer hard drives, solid state drives, removable computer data storage media, and cloud-based data storage systems. A preferred repository is structured to allow access to all of the detector array data from any time frame during which detector array data was collected, including data in real time or nearly real time.

The detector array data comprises air sample analyte information, time at which each air sample was obtained, wind speed (or wind velocity) and direction at each time at which an air sample was obtained, and information indicative of the location of the detector array which collected the data. The detector array data in the repository can be reviewed as convenient. More preferably the detector array data can be viewed in real time. It is to be understood that real time includes nearly real time, as there may be slight delays in analyzing the sample and transmitting the data.

Air sample analyte information includes the identity of the analyte and its concentration. The word “time,” as used throughout this document, unless otherwise specified, includes the (calendar) date. The location of the detector array also serves as the location for each co-located air analyzer and anemometer, and the location at which the air sample is obtained. In some embodiments, the location of the detector array is expressed relative to a fixed point near one or more of the detector arrays. In preferred embodiments, the location of each detector array is expressed as a geographic position.

In some embodiments, the location at which the air sample is obtained includes the height of the air intake, i.e., the height at which the air sample is obtained.

The system includes a data processor having machine readable logic instructions which when executed by a central processing unit processes at least a portion of the detector array data so as to generate one or more signals convertible into one or more display images showing the detector array data in human-perceivable form. In preferred embodiments, the system is set up to allow access to the detector array data in real time, so that the data provide real time wind direction or directions around the air analyzer(s) which detected the substance(s).

The computer programs employed in the one or more central processor(s) that are operatively in communication with the data repository and generate output that can be transmitted to the display of the system are typically written in one or more of a variety of computer languages and compiled into an executable form suitable for execution by the processor(s) during use. The computer software program languages are usually one or more of SQL, C, Javascript and HTML, and in one embodiment, the computer software program languages are a combination of SQL, C, Javascript and HTML).

The detector array data can be viewed for a single location taken over time, or for multiple locations at one time or at one or more points in time. Preferably, the detector array data is viewed for multiple locations over a period of time; typically, the multiple locations are in the same, usually contiguous, area. The detector array data can, in real time, as well as over one or more selected periods of time, provide a record that displays, preferably graphically, a wind direction for the substance(s) detected and the corresponding concentration(s) of the detected substance(s).

In some embodiments, the display images showing the detector array data in human-perceivable form are in the form of a spreadsheet, table, or one or more graphs. One way of displaying the detector array data in graph format is one graph per detector array, with time as the x axis, and separate lines (and scales) on the y axis for the analyte concentration and the wind speed, optionally with an indication of a desired limit or threshold for the analyte concentration shown on the graph.

In other embodiments, the display images showing the detector array data in human-perceivable form are in the form of a graphical representation; preferably, the graphical representation is a vector. Generally, each vector displayed indicates wind speed and direction at the time air sample was obtained.

In preferred embodiments, the vectors display the concentration(s) of the substance(s) in the air sample that was obtained and analyzed, the wind speed and direction, and the time the air sample was obtained. The vector representation preferably shows the direction of travel to the air intake of the air analyzer. The vector display aids in the determination of the source of the analyte(s) being measured, and may indicate proximity to a point of concern, e.g., a fence line of an industrial facility. To determine sources of emissions, the data over time, including wind speed and direction, address the problem of wind variability, and allow the vectors to indicate a possible source or sources of emissions. In some preferred embodiments, the graphical representations, preferably vectors, are displayed on a map of the area in which the detector array(s) are located, where the map is a street map, aerial map, or georeferenced aerial or satellite photograph which indicates the location of each detector array.

In some embodiments in which the display images are in the form of a graphical representation, the graphical representation accounts for obstacles present in the wind direction at the height of the air intake, which normally limits the length of the vector from the point of the obstacle at the height of the air intake. Obstacles are obstructions such as pipelines, storage tanks, walls, and buildings.

In preferred embodiments in which the display images are in the form of a graphical representation, the detector array data can be displayed differently to indicate that a pre-selected value for one or more air analyte concentrations is being approached, and/or has been reached or exceeded, to be displayed; preferably, the graphical representation is part of a vector display. In some preferred embodiments, for each set of detector array data collected, a color-coded vector is drawn showing the direction, in degrees, of the wind toward the air intake. More preferably, each of the colors represents a different concentration range for the analyte(s) being measured. The air analyte concentration ranges for each vector color can be selected to conform to ranges of interest; for example, a range may be selected in which the upper limit is at or near a regulatory limit for the substance, or when the air analyte concentration has exceeded the upper limit that the air analyzer can quantify.

In another preferred embodiment, display images of the detector array data from a selected set of time are displayed in chronological sequence; preferably, the detector array data from the selected set of time can be paused or moved forward and/or backward along the chronological sequence; more preferably, the graphical representation is a vector. Selecting any time and one or more detector array location(s) will display the detector array data, preferably in an animated manner, for all of the detector array data taken over a selected time period in chronological order for the selected detector array location(s). Preferably, one or more substances can be selected when more than one substance is being monitored. More preferably, controls on the software allow the user to stop the display, reverse it, or run it forward in chronological sequence. In some preferred displays, when detector array data is being played over time in a chronological sequence, all of the data remains on the display (“trail”), or, more preferably, the earlier data begins to fade (“ghost”), while more recent data is relatively bright.

In a particularly preferred embodiment, the detector array data for one or more selected analysis location(s), preferably an area encompassed by a system of detector arrays, is displayed on a street map, aerial map, or georeferenced aerial or satellite photograph which indicates the location of each detector array that is part of the selected analysis location(s).

In other preferred embodiments in which the display images are in the form of a graphical representation, the display images can indicate that a pre-selected value for one or more analytes is being approached or has been reached or exceeded to be displayed; preferably, the graphical representation is part of a vector display, and the display images of the detector array data from a selected set of time are displayed in chronological sequence; preferably, the detector array data displayed from the selected set of time can be paused or moved forward and/or backward along the chronological sequence; more preferably, the graphical representation is a vector.

In some preferred embodiments, the display, preferably in graphical representations, when a cursor hovers above a particular representation (preferably a vector) on the display, the cursor hover (or “mouseover”) elicits a pop-up display of the detector array data for the specific detector at the selected time on the display. Preferably, a pop-up display is available when at least a portion of the detector array data is being played over time in a chronological sequence, and when the chronological sequence is stopped at a selected time.

In particularly preferred embodiments, the systems and methods of this invention provide a combination of quantitative analysis of one or more substances (air sample information) with concurrent anemometer data collected in a repository connected to the internet, and an application that displays directional vectors for each set of detector array data over a selected area, often a geographically-defined area, over time.

In the methods for assembling a system of the invention, an anemometer can be co-located with an already-placed air analyzer, or an air analyzer and an anemometer can both be placed in a co-located position. In some instances, there is a combination of placing one or more anemometers co-located with one or more already-placed analyzers and placing one or more air analyzers and one or more anemometers co-located positions. When a transmitter is not present with the air analyzer or anemometer, a transmitter is connected to the air analyzer and/or anemometer as needed.

The detector arrays are typically placed within the grounds of an industrial facility such as a chemical plant or refinery, or near population areas or around environmental releases (e.g., storage tank ruptures or train wrecks) to determine the source of emissions, so that the source of emissions can be addressed to minimize or stop the leaking and/or to determine if emissions are traveling toward sensitive populations or facilities.

The air intakes, air analyzers, anemometers, transmitter for transmitting detector array data, repositories, data processor, and preferences therefor, are as described above.

In the methods of the invention for obtaining vectored air analyte data, each time an air sample is analyzed, the repository records and stores the following data: information indicative of the location of the detector array; the time at which the air sample was obtained; air sample analyte information comprising the identity and amount (concentration) of the substance(s); the wind speed (usually in miles or kilometers per hour), and the wind direction in degrees, preferably relative to true north. Preferably, correction to true north from magnetic north is performed on every set of detector array data collected. An algorithm supplied by the National Oceanic and Atmospheric Administration (NOAA) through their National Centers for Environmental Information referred to as “Magnetic Field Calculators” is suitable for this correction. The information indicative of the location of the detector array may be an identifier for the specific detector array that is generating the detector array data; because the detector arrays are in fixed positions, this information does not need to be re-measured for each air sample.

In the methods of the invention, air sampling and data collection preferably occur at a pre-set frequency, e.g., every minute, every ten minutes, every thirty minutes, every hour, but can be initiated by manual command. Air sampling and data collection can occur at any time of day or night as desired.

The detector array data can be displayed on paper or on a computer screen; in preferred embodiments, the detector array data is displayed on the screen of a mobile device; more preferably, the detector array data is displayed on the mobile device screen in real time.

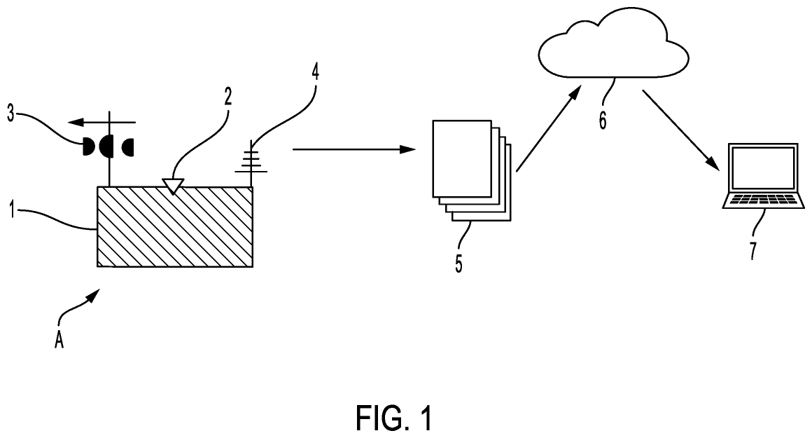

FIG. 1 shows a preferred system of the invention, including a detector array A comprising i) an air analyzer 1 comprising an air intake 2 and ii) an anemometer 3 co-located with the air analyzer 1. Also shown is a transmitter in the form of an internet-connected wireless transmitter for transmitting detector array data 4, detector array data 5, a preferred repository in the form of a storage medium 6, and a display means in the form of a computer screen 7. It will be appreciated that the storage medium 6 typically will be in communication with a central processing unit or other data processor for retrieving array data 5 and processing such data in order to general output responsive to commands or queries generated by a user interfacing with the system via a computer or hand-held device that is in secure communication with the data processor associated with medium 6. Computer screen 7 can be a component part of a personal computer or a mobile device such as a cell phone, laptop or other hand-held device capable of receiving data signals over an internet connection (typically via a cellular or wireless network connection) using an internet protocol or other communication protocol and executing, on a processor within the mobile device or personal computer, a software application that facilitates processing of the output signals to generate a display and interface between the user and the rest of the system.

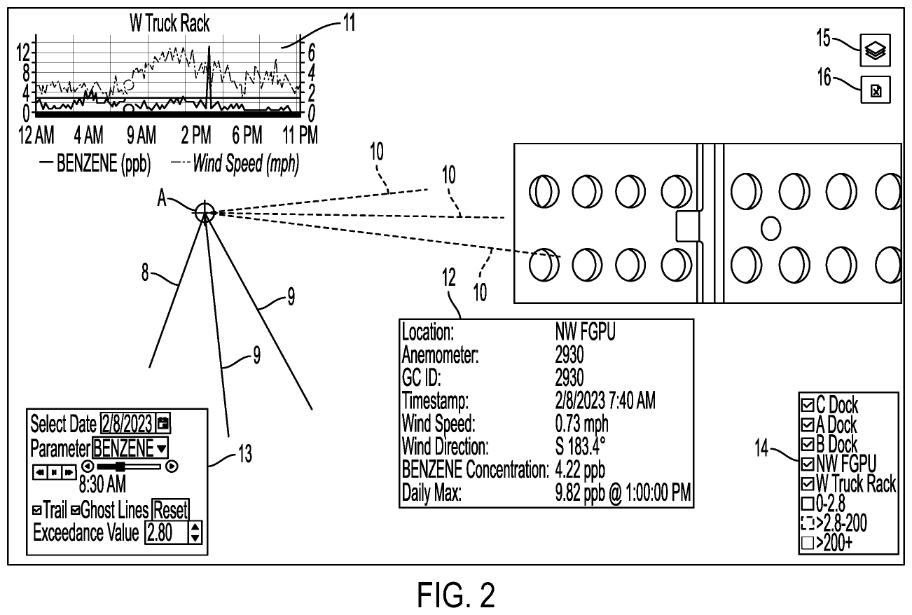

FIG. 2 shows a view of computer screen 7 of FIG. 1 as a display for the air analyte data. In FIG. 2, a detector array A is shown with more recent vectors 9 darker or brighter in color than earlier-in-time vectors 8; vectors 8 and 9 may be a color (e.g., aqua or blue) to indicate that the analyte concentration is below a pre-set value, while vectors 10 may be a color (e.g., red) to indicate that the analyte concentration meets or exceeds a pre-set value. Graph 11 shows the pre-set value for the analyte concentration as a horizontal straight line, the analyte concentration as wavy line below the horizontal straight line, and the wind speed as a wavy line mostly above the horizontal straight line. In preferred displays, preferably in graphical representations, when a cursor hovers above a particular representation (preferably a vector) on the display, the cursor hover (or “mouseover”) elicits a pop-up display 12 of the detector array data for the specific detector at the selected time on the display. Rectangular item 13 represents a detector array data play back controller that allows at least a portion of the detector array data to be displayed over time in a chronological sequence, and to stop and/or reverse the chronological sequence playback, and also allowing selection of a time period for which detector array data is to be played. Rectangle 14 represents a computer menu allowing the selection of one or more detector arrays to be displayed, and allowing the data to be displayed differently for different concentration ranges for the air analyte data. Square 15 represents a computer menu generated by the data processor (as a button) that permits selection of a type of map on which to overlay the graphical representation of the detector array data, where the type of map can be a street map, aerial map or photograph, or satellite photograph. Square 16 represents a computer menu option (as a button) allowing the air analyte data to be displayed and/or downloaded and as a spreadsheet. The unlabeled rectangles encompassing circles represent a set of storage tanks at a chemical plant.

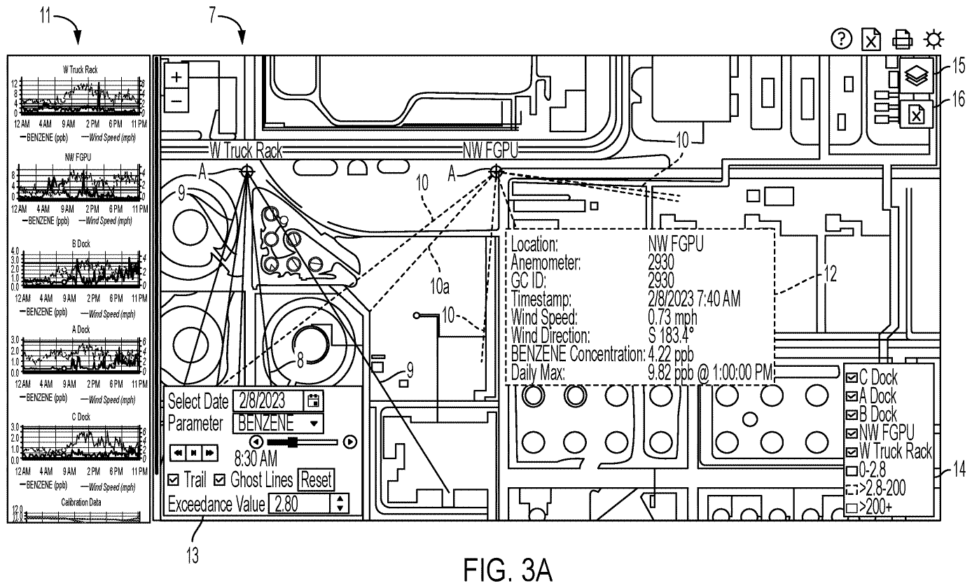

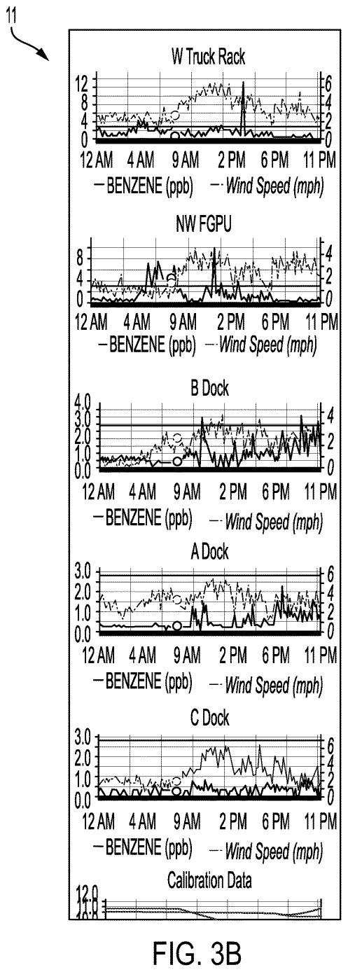

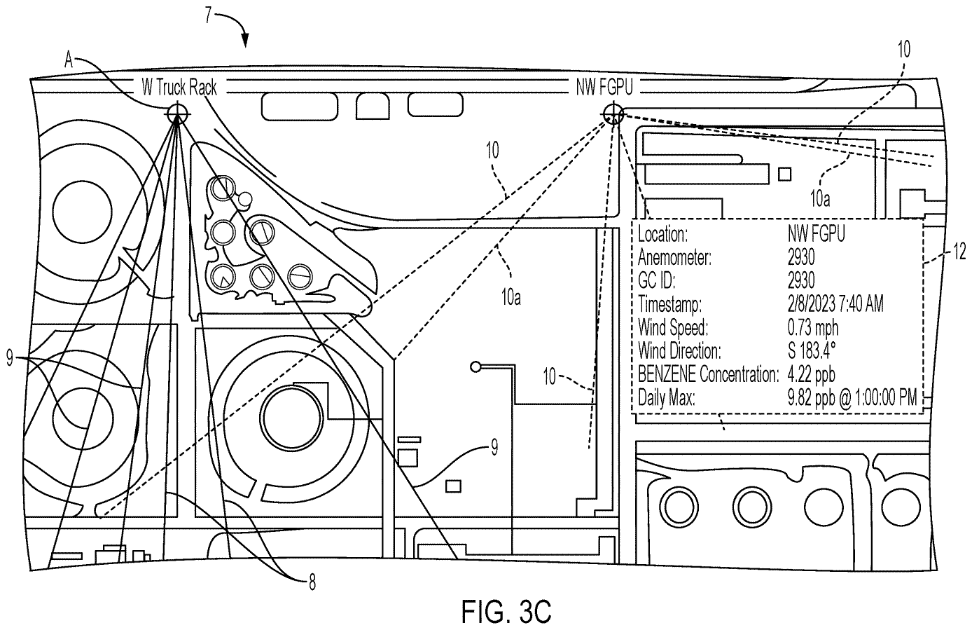

FIG. 3A shows a preferred view of computer screen 7 of FIG. 1, displaying a representation of air analyte data over a period of time for two detector arrays A at different locations overlaid on an aerial photograph of the facility where the detector arrays are located; the series of graphs 11 at the left of the Figure are for a specific substance at several detector array locations. Similar to FIG. 2, each graph 11 shows the pre-set value for the analyte concentration as a horizontal straight line, the analyte concentration as wavy line below or mostly below the horizontal straight line, and the wind speed as a wavy line often above the horizontal straight line. Each graph is for a different detector array location, except for the bottom graph, which displays calibration data. Two detector arrays A at different locations are shown in FIG. 3A; vectors 8 and 9 are to the detector array A on the left, while vectors 10 and 10a are to the detector array A on the right. The vectors 10 and 10a may be a color (e.g., red) that indicates that an analyte concentration limit or threshold value set by the user has been exceeded, while vectors 8 and 9 may be a color (e.g., blue or green) to indicate that the analyte concentration is below the limit or threshold value set by the user. The vectors 8 and 9 are shown with more recent vectors 9 darker or brighter in color than earlier-in-time vectors 8; the vectors are 10 and 10a shown with more recent vectors 10 darker or brighter in color than earlier-in-time vectors 10a. To the right of the bottom graph is a rectangular item 13 representing a detector array data playback controller similar to rectangular item 13 of FIG. 2, and preferably having the features of the playback controller described in FIG. 2. In the bottom right corner, FIG. 3A displays a computer menu 14 allowing the selection of one or more detector arrays to be displayed, and allowing the data to be displayed differently for different concentration ranges for the air analyte data, similar to rectangle 14 in FIG. 2. FIG. 3A has two small squares in the upper right corner, one square 15 representing a computer menu generated by the data processor (as a button) that permits selection of a type of map on which to overlay the graphical representation of the detector array data, where the type of map can be a street map, aerial map or photograph, or satellite photograph, and the other square 16 representing a computer menu option (as a button) allowing the air analyte data to be displayed and/or downloaded and as a spreadsheet, similar to squares 15 and 16 in FIG. 2. The text-containing rectangle 12 near the middle of FIG. 3A is a pop-up display caused by cursor hover (or “mouseover”) of the detector array data for the specific detector array's vector at the selected time on the display, similar to 12 in FIG. 2.

FIG. 3B shows is an enlargement of the series of graphs 11 shown in FIG. 3A. Each of these graphs is for the same analyte at different detector array locations over the same time period and are similar to graph 11 shown in FIG. 2. In FIG. 3B, each graph 11 shows the pre-set value for the analyte concentration as a horizontal straight line, the analyte concentration as wavy line below or mostly below the horizontal straight line, and the wind speed as a wavy line often above the horizontal straight line. Each graph is for a different detector array location, except for the bottom graph, which displays calibration data.

FIG. 3C shows an enlargement of the vectors of FIG. 3A overlaid on the aerial photograph of a chemical plant where the detector arrays A are located, as shown in FIG. 3A. In FIG. 3C, there is a pop-up display 12 (light-colored rectangle) similar to the pop-up display 12 in FIG. 2. The vectors 8 and 9 in FIG. 3C from detector array A on the left are shown with more recent vectors 9 darker or brighter in color than earlier-in-time vectors 8; vectors 8 and 9 may be a color (e.g., blue or green) to indicate that the analyte concentration is below the limit or threshold value set by the user. The vectors from the detector array A on the right are shown with more recent vectors 10 darker or brighter in color than earlier-in-time vectors 10a; vectors 10 and 10a may be a color (e.g., red) that indicates that an analyte concentration limit or threshold value set by the user has been exceeded. The vectors 8, 9, 10, and 10a are shown overlaid on the aerial photograph of the chemical plant. Depending on the height of the detector array, the vectors may not account for obstacles present in the wind direction at the height of the air intake; the left-most vector is shown extending over a storage tank (white circle).

As will now be appreciated, systems and methods of the invention may be used to analyze air analytes present in a variety of geographic locations and facilities where it is desirable to monitor ambient air for analytes of interest over time, to facilitate a rapid determine of their directional source and concentration over a target area.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

The invention may comprise, consist, or consist essentially of the materials and/or procedures recited herein.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about”, the claims include equivalents to the quantities.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

This invention is susceptible to considerable variation in its practice. Therefore the foregoing description is not intended to limit, and should not be construed as limiting, the invention to the particular exemplifications presented hereinabove.

1. A system for obtaining vectored air analyte data, which system comprises:
one or more detector arrays, each detector array having a location and each detector array comprising:
i) an air analyzer comprising an air intake configured to obtain respective air samples over time, the air analyzer being configured to analyze the air samples obtained over time by the air intake to produce air sample analyte information,
ii) an anemometer co-located with the air analyzer, the anemometer being configured to measure wind velocity and wind direction concurrent with obtaining the air samples, and
iii) a transmitter for transmitting detector array data comprising the air sample analyte information, time at which each air sample was obtained, wind speed and wind direction at each time at which each air sample was obtained, and information indicative of the location of the detector array;
a repository in which the detector array data are stored after receipt from the transmitter; and
a data processor having machine readable logic instructions which when executed by a central processing unit processes at least a portion of the detector array data so as to generate one or more signals convertible into one or more display images showing the detector array data in human-perceivable form, wherein the display images showing the detector array data in human-perceivable form displays the detector array data in the form of a graphical representation, and wherein the graphical representation is a vector.

2. The system as in claim 1 wherein:
the air analyzer comprises one or more substance-specific sensors, optionally wherein the substance-specific sensor is selected from a carbon monoxide sensor, a carbon dioxide sensor, a methane sensor, and/or a particulate sensor; or
wherein the air analyzer is a multi-substance analyzer, optionally wherein the multi-substance analyzer is a gas chromatograph or multi-substance sensor.

3. (canceled)

4. The system as in claim 1 wherein:
the transmitter for transmitting detector array data comprises a wireless transmitter configured to transmit the detector array data to a global computer network in communication with the repository for storage therein; and/or
wherein the repository is a computer hard drive, solid state drive, removable computer data storage medium, or a cloud-based data storage system.

5. The system as in claim 1 wherein the information indicative of the location of the detector array is a geographic position, and/or wherein the wind direction is corrected from magnetic north to true north.

6-7. (canceled)

8. The system as in claim 1 wherein the display images showing the detector array data in human-perceivable form may also include are a spreadsheet, table or one or more graphs, or a graphical representation other than a vector.

9. The system as in claims 1 wherein the location includes the height of the air intake.

10. The system as in claim 9 wherein:
the display images showing the detector array data in human-perceivable form are a graphical representation, and wherein the graphical representation accounts for obstacles present in the wind direction at the height of the air intake;
the display images showing the detector array data in human-perceivable form has an indication that a pre-selected value for at least a portion of the air sample analyte information has been reached or exceeded; and/or
the display images showing the detector array data in human-perceivable form displays the detector array data from a selected set of time in sequence, optionally wherein the detector data from the selected set of time can be paused or moved forward and/or backward along the sequence.

11-13. (canceled)

14. A method for assembling a system for obtaining vectored air analyte data, which method comprises:
forming one or more detector arrays, each detector array having a location, by placing an anemometer co-located with an air analyzer,
each anemometer being configured to measure wind velocity and wind direction concurrent with obtaining an air sample, and
each air analyzer comprising an air intake configured to obtain respective air samples over time, each air analyzer being configured to analyze the air samples obtained over time by the respective one of the air intakes to produce air sample analyte information, and
connecting a transmitter for transmitting detector array data comprising the air sample analyte information, time at which each air sample was obtained, wind speed and wind direction at each time at which each air sample was obtained, and information indicative of the location of the detector array;
connecting a repository in which the detector array data are stored from the transmitter for transmitting detector array data; and
connecting to the repository a data processor having machine readable logic instructions which when executed by a central processing unit processes at least a portion of the detector array data so as to generate one or more signals convertible into one or more display images showing the detector array data in human-perceivable form, wherein the display images showing the detector array data in human-perceivable form displays the detector array data in the form of a graphical representation, and wherein the graphical representation is a vector.

15. The method as in claim 14 also comprising placing one or more air analyzers and a transmitter with the anemometer.

16. The method as in claim 14 wherein:
the air analyzer comprises one or more substance-specific sensors, optionally wherein each substance-specific sensor is selected from a carbon monoxide sensor, a carbon dioxide sensor, a methane sensor, and/or a particulate sensor; or
the air analyzer is a multi-substance analyzer, optionally wherein the multi-substance analyzer is a gas chromatograph or multi-substance sensor.

17. (canceled)

18. The method as in claim 14 wherein:
the transmitter for transmitting detector array data comprises a wireless transmitter configured to transmit the detector array data to a global computer network in communication with the repository for storage therein; and/or
the repository is a computer hard drive, solid state drive, removable computer data storage medium, or a cloud-based data storage system.

19. The method as in claim 14 wherein the geographic position of each of the air analyzers and anemometers is determined by global positioning satellite information, with correction from magnetic north to true north.

20. The method as in claim 14 wherein the information indicative of the location of the detector array is a geographic position, and/or wherein the wind direction is corrected from magnetic north to true north.

21-22. (canceled)

23. The method as in claim 14 wherein the display images showing the detector array data in human-perceivable form may also include a spreadsheet, table or one or more graphs, or a graphical representation other than a vector.

24. The method as in claim 14 wherein the location includes the height of the air intake.

25. The method as in claim 24 wherein:
the display images showing the detector array data in human-perceivable form are a graphical representation, and wherein the graphical representation accounts for obstacles present in the wind direction at the height of the air intake;
the display images showing the detector array data in human-perceivable form has an indication that a pre-selected value for at least a portion of the air sample analyte information has been reached or exceeded; and/or
the display images showing the detector array data in human-perceivable form displays the detector array data from a selected set of time in sequence, optionally wherein the detector data from the selected set of time can be paused or moved forward and/or backward along the sequence.

26-28. (canceled)

29. A method for obtaining vectored air analyte data, which method comprises:
generating detector array data from a detector array comprised of i) an air analyzer, ii) an anemometer co-located with the air analyzer, and iii) a transmitter, the generating comprising:
obtaining an air sample with an air intake comprising part of the air analyzer, the air intake configured to obtain respective air samples over time, the air analyzer being configured to analyze the air samples obtained over time by the air intake to produce air sample analyte information;
analyzing the air sample obtained by the air analyzer to produce air sample analyte information;
measuring wind velocity and wind direction with the anemometer concurrent with obtaining the air sample;
wherein the detector array data comprises the air sample analyte information, time at which each air sample was obtained, wind speed and wind direction at each time at which each air sample was obtained, and information indicative of the location of the detector array;
transmitting the detector array data with the transmitter for transmitting detector array data;
storing the detector array data in a repository after receipt from the transmitter; and
processing at least a portion of the detector array data from the repository into a human-perceivable form via a data processor having machine readable logic instructions which when executed by a central processing unit processes at least a portion of the detector array data so as to generate one or more signals convertible into one or more display images showing the detector array data in human-perceivable form, wherein the display images showing the detector array data in human-perceivable form displays the detector array data in the form of a graphical representation, and wherein the graphical representation is a vector.

30. The method as in claim 29 wherein:
the air analyzer comprises one or more substance-specific sensors, optionally wherein the substance-specific sensor is selected from a carbon monoxide sensor, a carbon dioxide sensor, a methane sensor, and/or a particulate sensor; or
the air analyzer is a multi-substance analyzer, optionally wherein the multi-substance analyzer is a gas chromatograph or multi-substance sensor.

31. (canceled)

32. The method as in claim 29 wherein:
the transmitter for transmitting detector array data comprises a wireless transmitter configured to transmit the detector array data to a global computer network in communication with the repository for storage therein; or
the repository is a computer hard drive, removable computer data storage medium, solid state drive, or a cloud-based data storage system.

33. The method as in claim 29 wherein the information indicative of the location of the detector array is a geographic position, and/or wherein the wind direction is corrected from magnetic north to true north.

34-35. (canceled)

36. The method as in claim 29 wherein the display images showing the detector array data in human-perceivable form may also include are a spreadsheet, table or one or more graphs, or a graphical representation than a vector.

37. The method as in claims 29 wherein the location includes the height of the air intake.

38. The method as in claim 37 wherein:
the display images showing the detector array data in human-perceivable form are a graphical representation, and wherein the graphical representation accounts for obstacles present in the wind direction at the height of the air intake;
the display images showing the detector array data in human-perceivable form has an indication that a pre-selected value for at least a portion of the air sample analyte information has been reached or exceeded; and/or
the display images showing the detector array data in human-perceivable form displays the detector array data from a selected set of time in sequence, optionally wherein the detector data from the selected set of time can be paused or moved forward and/or backward along the sequence.

39-41. (canceled)
